(19) United States
(12) Patent Application Publication
(10) Pub. No.: US 2005/0175675 A1
(43) Pub. Date: Aug. 11, 2005

(54) Film-shaped, dissolvable preparations for active substance release and method for the production thereof

(76) Inventor: Frank Seibertz, Rheinbrohl (DE)

Correspondence Address: D Peter Hochberg;Baker Building, 6th Floor, 1940 East 6th Street, Cleveland, OH, 44114, US

(21) Appl. No.: 10/517,093

(22) PCT Filed: May 8, 2003
(86) PCT No.: PCT/EP03/04806

(30) Foreign Application Priority Data
Jun. 4, 2002 (DE) 102 24 607.6

(51) Int. Cl. A61K 9/70

(52) U.S. Cl. 424/443

(57) ABSTRACT

A film-shaped preparation disintegratable in aqueous media for administration of substances to the human or animal body. The preparation contains at least one water-soluble polymer. The preparation contains one or more components that produce a gas upon action of moisture, being in the presence of an aqueous medium or by a temperature change.

### BACKGROUND OF THE INVENTION

### CROSS REFERENCE TO RELATED APPLICATIONS

This application is a National Stage application of International Application No. PCT/EP03/04806, filed on May 8, 2003, which claims priority of German application number 102 24 607.6, filed on Jun. 4, 2002.

1. Field of the Invention

The invention relates to film-shaped preparations which are disintegratable in aqueous media and which are produced on the basis of water-soluble polymers and can be used for administering substances to the human or animal body. The invention further relates to processes for the production of such preparations as well as to the use thereof as pharmaceutical administration forms.

2. Description of the Prior Art

Flat-shaped administration forms, especially oral administration forms, which disintegrate in an aqueous environment and enable a quick release of active substances in the oral cavity or in other apertures or cavities of the body are already known. Such administration forms can be configured in the form of “wafers”, for example. Due to their small layer thickness and disintegratability or dissolvability these film-shaped, flat administration forms are particularly suitable for the quick release of medicaments and other active substances in the oral cavity.

Flat-shaped, wafer-like films for delivering substances to the human or animal body are as a rule made of film-forming, water-soluble polymers. When these wafers come into contact with water or with a body fluid (e.g. saliva), the polymers dissolve, thus releasing the active substance. The quicker the aqueous liquid reaches the inner regions of the wafer, the quicker the wafer disintegrates. Therefore, the disintegration rate decreases with increasing layer thickness.

The thickness of such systems is in turn determined to a large extent by the amount and type of the active substance to be guided. Thick wafers have the unpleasant property of sticking to the palate because of their flat shape and their retarded disintegration. This is due, on the one hand, to the polymer layers dissolving superficially, thus forming a slushy and sticky film, and, on the other hand, to the administration forms adhering to the palate upon contact with the oral mucosa because of the pressure gradient from the upper side to the lower side of the palate.

The property of these film-shaped administration forms of sticking to the palate and other surfaces of the oral mucosa may cause an unpleasant sensation in the persons concerned or in the patients, i.e. the “mouthfeel” caused by the wafers is unpleasant or disturbing and therefore in need of improvement.

### SUMMARY OF THE INVENTION

An object of the present invention is to provide a film-shaped preparation of the afore-mentioned type which has the known advantages of quickly disintegrating administration forms but which at the same time has improved disintegration properties resulting in an accelerated active substance release.

It is an additional object to reduce the tendency of adhering or sticking to the oral mucosa, so that an improved “mouthfeel” results if the preparation is used as an oral administration form.

A further object is to indicate processes by means of which film-shaped preparations having the aforementioned improved properties may be obtained.

The objects of the present invention are surprisingly achieved by providing a film-shaped preparation which is disintegratable in an aqueous media and contains at least one water-soluble polymer and which additionally contains one or two components that produce a gas upon action of moisture, being in the presence of an aqueous medium or if a change in temperature occurs. This gas is released from the preparation if it is placed in the oral cavity, for example, and comes into contact with saliva. The formation of gas bubbles during oral intake of a film-shaped preparation (“wafer”) strongly affects the internal structure of the wafer and causes it to rapidly disintegrate into a plurality of fragments. The wafer is thus virtually blown up by the gas bubbles forming in its interior. This results in an abrupt increase in the available surface, which leads to an accelerated release of active substance.

In addition, the fact that gas bubbles escape at the surfaces of the wafer prevents the wafer from sticking to the mucosa. This in turn assists the supply of further liquid to both sides of the wafer. By contrast, in the case of the system adhering to the mucosa, as may occur in conventional wafers, only one side would be available for increased liquid absorption.

In the case of oral application of the inventive wafer the escape of gas bubbles at the surfaces of the wafer in addition causes an improved “mouthfeel” since the wafer cannot adhere to the oral mucosa or tongue but is continuously being separated from the mucosa by the bursting of the bubbles.

The object is furthermore achieved by the production processes described in claims 13 to 16. Said claims enable the production of film-shaped preparations according to the main claim which are disintegratable in aqueous media. An essential advantage of these methods is that during the production of the preparations it is possible to add gas-forming components which can be activated by water or moisture, without the occurrence of premature gas formation while the preparation is being manufactured.

### DETAILED DESCRIPTION OF THE INVENTION

The preparations according to this invention are flat-shaped films containing at least one water-soluble polymer. The water-soluble polymer(s) form(s) the basic structure, also called a matrix, of the preparation. The composition of the matrix may further comprise auxiliary substances of a very different type by means of which the chemical and physical properties of the films are additionally influenced. The weight content of the water-soluble polymer(s) is preferably in the range of 10 to 95%-wt, especially preferably in the range of 15 to 70%-wt, in each case relative to the entire preparation (dry matter).

The matrix-forming polymer components used for this purpose are water-soluble or at least partially water-soluble; they may be thermoplastic or non-thermoplastic. In the manufacture of the film-shaped preparations, thermoplastic formulations can be extruded under heat, whereas non-thermoplastic polymers can be extruded as high-viscous solutions.

Partial water-solubility is understood to mean the property of a large number of polymers of being swellable in water or in aqueous media. The water molecules in this case enter the polymer material slowly, which results in swelling and the formation of a gel. Subsequent disintegration of the swollen gel to a true solution does not occur. This is true, in particular, with polymers of very high molecular weight or with cross-linked polymers.

The following polymers may be used for this purpose: polyvinyl alcohol (PVA), polyethylene oxide, copolymer of methyl vinyl ether and maleic acid, cellulose derivatives such as hydroxypropyl methyl cellulose (HPMC), hydroxypropyl cellulose (HPC), sodium-carboxymethyl cellulose (NaCMC), methyl cellulose (MC), hydroxyethyl cellulose (HEC), hydroxypropyl ethyl cellulose (HPEC), starch and starch derivatives, gelatins, polyvinyl pyrrolidone (PVP), gum arabic, pullulan or acrylates. Mixtures of two or more of the aforementioned polymers may also be used.

As auxiliary substances, which are in principle known to those skilled in the art, it is possible to use substances from the following groups: Fillers such as SiO₂; colorants such as quinoline yellow or TiO₂; disintegrants, respectively wicking agents, which draw water into the matrix and explode the matrix from within, e.g. aerosil; emulsifiers such as Tween (polyethoxylated sorbitan fatty acid ester), Brij (polyethoxylated fatty alcohols); sweeteners such as aspartame, sodium cyclamate and saccharine; softeners such as PEG (polyethylene glycol) or glycerol; preservatives such as, for example, sorbic acid or its salts. The content of these additives may preferably amount to up to 30%-wt, in particular 1 to 20%-wt, each relative to the entire preparation.

Components which upon action of moisture or in the presence of an aqueous medium or in the case of a change in temperature produce a gas are in principle known to those skilled in the art. Preferably, the film-shaped preparations according to the present invention contain one or more gas-forming component(s) selected from the group comprising carbonates, especially sodium carbonate, ammonium carbonate, magnesium carbonate, potassium carbonate and hydrogen carbonate, especially sodium hydrogen carbonate, and acids, especially carboxylic acids such as citric acid, malic acid, acetic acid, lactic acid, fumaric acid, gluconic acid, tartaric acid, as well as acid regulators, especially salts of acetic acid, sodium dihydrogen phosphate or disodium hydrogen phosphate, sodium tartrate, sodium ascorbate.

Gas formation is preferably caused by combining two or more components, especially by combining an alkaline earth carbonate or alkaline metal carbonate or alkaline metal hydrogen carbonate with a carboxylic acid. Suitable carbonates or hydrogen carbonates are, in particular, sodium hydrogen carbonate, sodium carbonate, potassium carbonate and potassium hydrogen carbonate. Carboxylic acids which may be used are citric acid, malic acid, acetic acid, lactic acid, fumaric acid, gluconic acid and tartaric acid. A combination of sodium hydrogen carbonate with citric acid is especially preferred.

The proportion of the gas-forming component(s) may be up to 70%-wt, relative to the dry matter of the preparation; preferably, the content is in the range from 5 to 60%-wt.

In the case of the above-mentioned gas-forming components, the gas formed upon action of an aqueous medium is carbon dioxide. This, however, does not exclude the possibility of another gas, e.g. nitrogen, forming when a different gas-forming compound or a mixture of compounds is used. If the gas formed in the presence of water or an aqueous medium is CO₂, this results in a further advantage of the inventive film-shaped administration forms in that there is an improved absorption of the active substance in the body. According to a preferred embodiment, the preparations of the invention are characterized by forming an acidic environment in the presence of water or of an aqueous medium.

Apart from the above-mentioned components involved in the formation of the gas, the film-shaped preparations may additionally contain one or more acid regulators. Suitable for this purpose are particularly salts of acetic acid, sodium dihydrogen phosphate or disodium hydrogen phosphate, sodium tartrate and sodium ascorbate.

Activation of the gas formation preferably takes place under action of water, moisture or an aqueous medium. The film-shaped preparations may furthermore be configured such that gas formation is activated by a change in the thermal conditions, for instance if a film-shaped preparation is taken orally and in the process is heated under the influence of the body heat (e.g. if its temperature rises to above 25 to 35° C.). Gas formation by thermal activation is achieved by using blowing agents, amongst which are ammonium carbonate, ammonium hydrogen carbonate, hartshorn salt (a mixture of ammonium carbonate, ammonium hydrogen carbonate and ammonium carbamate), as well as hydrogen carbonate (sodium hydrogen carbonate, potassium hydrogen carbonate) in mixture with acid phosphates, acid pyrophosphates, citric acid or tartaric acid.

Another possibility which may be advantageous is activating the gas formation by a change in the pH value, for example after oral administration of a film-shaped preparation. The access of moisture upon contact with saliva enables the reaction between the gas-forming components (carbonate and/or hydrogen carbonate on the one hand, and acid component on the other). The CO₂-releasing reaction is due to an acidification (pH activation) of the carbonate or hydrogen carbonate component.

In the context of the present invention “aqueous media” is understood to mean, in particular, water, aqueous solutions, suspensions, dispersions, aqueous solvent mixtures as well as physiological liquids or body fluids (e.g. secretions of the body, saliva, mucus).

Due to their gas forming capacity, the film-shaped preparations according to the present invention stand out for their improved disintegration properties. These preparations are preferably configured as rapidly disintegrating films, that is, they have disintegration times in the range from 1 s to 5 min, preferably in the range from 1 s to 1 min, especially preferably in the range from 1 s to 30 s.

The film-shaped preparations may have a thickness in the range from 5 μm to 3 mm, preferably between 10 μm and 1 mm, and particularly preferably between 20 μm and 500 μm.

The film-shaped preparations according to one embodiment of the present invention generally have a mono-layer structure. According to an alternative embodiment, the preparations may be made up of at least two layers connected with each other.

In one particular embodiment it is provided that the film-shaped preparations are swellable in water or in aqueous media. This is achieved by providing a content of one or more swellable substances, for example from the group comprising the hydrophile polyacrylates, hydrophile polymethacrylates, anionic or cationic hydrogels, agar, carboxymethyl cellulose, methyl cellulose, tragacanth, gelatine, and swellable ion exchange resins.

The film-shaped preparations are advantageously suitable for use as administration forms for administering pharmaceutical substances. For this reason, it is provided in a preferred embodiment that such a preparation contains a pharmaceutical active substance or a combination of two or more pharmaceutical active substances. The active substance(s) may be present in dissolved, dispersed, suspended or emulsified form.

Optionally, further releasable substances may be contained, e.g. flavouring or sweetening substances.

Suitable as active substance are, without reservation, those substances which have a therapeutic effect in humans or animals. These may originate from the following groups: agents for treating infections; virostatics; analgesics such as fentanyl, sufental, buprenorphine; anaesthetics; anorectics; active substances for treating arthritis and asthma, such as terbutaline; anticonvulsives; antidepressants; antidiabetics; antihistaminics; anti-diarrhoeal agents; agents against migraine; agents against itching, nausea and retching, motion and seasickness, such as scopolamine and ondansetron; antineoplastic agents; anti-Parkinson agents; antipsychotic agents; antipyretics; antispasmodics; anticholinergics; agents against ulcer, such as ranitidine; sympathomimetics; calcium channel blockers such as nifedipine; beta blockers; beta-agonists, such as dobutamine and ritodrine; antiarrhythmics; antihypertensive agents such as atenolol; ACE inhibitors such as enalapril; benzodiazepine agonists such as flumazenil; coronary, peripheral and cerebral vasodilators; substances stimulating the central nervous system; hormones; hypnotics; immunosuppressing agents; muscle relaxants; prostaglandins; proteins, peptides; psychostimulants; sedatives; tranquilizers.

Suitable active substances are further found in the active substance groups of the parasympatholytics (e.g. scopolamine, atropine, berlactyzine), of the parasympathomimetics, of the cholinergics (e.g. physostigmine, nicotine), the neuroleptics (e.g. chloropromazine, haloperidol), the monoamine oxidase inhibitors (e.g. tranylcypromine, selegiline), the sympathomimetics (e.g. ephedrine, D-nor-pseudoephedrine, salbutamol, fenfluramine), the sympatholytics and anti-sympathotonics (e.g. propranolol, timolol, bupranolol, clonidine, dihydroergotamine, naphazoline), the anxiolytics (e.g. diazepam, triazolam), the local anaesthetics (e.g. lidocaine), the central analgesics (e.g. fentanyl, sufentanil), the antirheumatics (e.g. indomethacin, piroxicam, lornoxicam), the coronary therapeutics (e.g. glycerol trinitrate, isosorbide dinitrate), the estrogens, gestagens and androgens, the anti-histaminics (e.g. diphenhydramine, clemastin, terfenadine), the prostaglandin derivatives, the vitamins (e.g. vitamin E, cholecalciferol), the cytostatics and the cardioactive glycosides such as digitoxin and digoxin, for example.

The film-shaped preparations according to the invention may also be used for releasing one or more flavouring substances such as menthol or lemon flavour in the oral cavity. The flavouring substance(s) may, however, also be present in the preparation in combination with one or more pharmaceutical active substances.

The active substance content or the content of flavouring agent(s) is preferably 0.1 to 50%-wt, with particular preference 1 to 25%-wt, in each case relative to the dry matter of a film-shaped preparation.

The invention further comprises processes enabling the manufacture of film-shaped preparations which are disintegratable in aqueous media and which contain gas-forming components and produce a gas under action of moisture or in the presence of an aqueous medium.

The processes according to the present invention are based on the basic principle, according to which, a coating compound is first prepared which contains matrix polymer(s), gas-forming substance(s), active agent(s), and/or flavouring agent(s), and optionally further auxiliary substances. The coating compound is subsequently coated onto a support and then dried.

In the manufacture of the inventive preparations it must be taken into consideration that in the manufacture of water-soluble (or water-disintegratable) films, as a rule, water or an aqueous medium is used as solvent. Since the gas-forming components are activated by water or moisture, the desired gas-forming reaction would occur prematurely during the production of a coating compound which contains the water-soluble matrix polymers and the gas-forming components.

In accordance with the invention, this problem can be solved by means of the measures described below:

According to a first process of production according to the invention, it is provided that the production of the coating compound which contains the components of the preparation including the gas-forming component(s) is carried through by dissolving or suspending the components in a solvent or a suspending agent which is substantially free from water, e.g. ethanol. This prevents the gas forming reaction from being triggered already during the production of the coating compound. After coating and drying, a water-soluble film remains which shows gas formation upon contact with moisture and exhibits the desired properties.

“Substantially free from water” means that the water content is less than 8%-wt, preferably less than 5%-wt, and particularly preferably less than 1%-wt.

A second inventive process of production provides that the production of the coating compound containing the components of the preparation including the gas-forming component(s) is carried through by melting the components. Subsequently, the molten coating compound is extruded onto a support (carrier layer), preferably using a slot die. The cooled film is then available for further processing. In this process variant, thermoplastic water-soluble polymers or polymer mixtures are used as matrix-forming polymers. Since no water is used in this process, a premature activation of the gas formation is not possible.

A further possibility of producing film-shaped preparations having the claimed properties is to initially produce two films from two coating compounds and subsequently combining the films, each coating compound containing only a single one of the components necessary for the formation of the gas. For example, an aqueous polymer solution with sodium hydrogen carbonate and a further aqueous polymer solution with citric acid are prepared first, and one water-soluble film each is produced from these solutions by spreading these masses onto respective process films serving as support (e.g. polyester film, polyethylene film or metal foil) and subsequent drying.

Since both components have to be present to activate the gas formation, no premature gas formation occurs during the production of the coating compounds, even if water or aqueous media are used as solvent or suspending agent. The gas-forming process can thus not take place since the components necessary for gas formation are initially present in separate films. Subsequently, the two films are united—e.g. by laminating—such that one film results. Only after the application has taken place (e.g. oral administration) does the film-shaped preparation absorb water and the two gas-forming compounds come into contact with each other, thus triggering the gas formation.

This production process comprises the following steps: First, a first coating compound is produced which contains a first gas-forming component and further components of the film-shaped preparation. This can be done by dissolving, suspending or dispersing said components in an aqueous solvent or suspending agent. A second coating compound is produced in an analogous fashion; this coating compound contains a second gas-forming component and further components of the film-shaped preparation. The first and second components are reaction partners necessary for a gas-forming reaction. Each of the two coating compounds is spread on a support and dried, thus forming a first and a second film. The two films are combined by laminating one film on the other.

A fourth inventive production process makes use of the measure of providing at least one of the gas-forming components, or a mixture of the gas-forming components, in micro-encapsulated form during the production of the coating compound(s). Such encapsulation prevents the gas-forming reaction during the preparation of the compound. Only upon, for example, oral intake of the film—under the conditions present in the oral cavity such as pH value or body temperature—will the gas-forming reaction be activated. Suitable processes and auxiliary substances for microencapsulating particles are known to those skilled in the art.

According to this process, a coating compound is prepared which contains the components of the preparation including the gas-forming components, with at least one of the gas-forming components being present in microencapsulated form. The coating compound can be prepared by dissolving, suspending or dispersing the components in a solvent or a suspending agent. As activation of the formation of the gas is prevented by the microencapsulation, it is also possible to use aqueous media as solvents or suspending agents in this process. Thereafter, the coating compound is spread on a support and dried.

The film-shaped, disintegratable preparations according to the invention are advantageously suitable for use as administration forms for administering pharmaceutical active substances for therapeutic purposes, especially for oral, rectal or vaginal administration.

The invention will be illustrated by means of the following examples of embodiments:

### EXAMPLE 1

EXAMPLE 1

| | | Proportion |
|---|---|---|
| | | Dry matter |
| No. | Component | %-wt |
| 1 | Ethanol | |
| 2 | PVP | 33% |
| 3 | Citric acid | 20% |
| 4 | NaHCO₃ | 35% |
| 5 | Menthol | 7% |
| 6 | Aspartame | 5% |

### EXAMPLE 2

EXAMPLE 2

| | | Proportion |
|---|---|---|
| | | Dry matter |
| No. | Component | %-wt |
| 1 | Ethanol | |
| 2 | HPC | 33% |
| 3 | Citric acid | 20% |
| 4 | NaHCO₃ | 30% |
| 5 | Lemon flavour | 12% |
| 6 | Aspartame | 5% |

Preparation of the compounds of Examples 1 and 2: No. 1 is provided first and then No. 2 is added thereto while stirring such that a 15% polymer solution results. Subsequently, Nos. 3, 5 and 6 are added and stirred until the mass is homogenous, thereafter No. 4 is added and this is stirred until the mass is homogenous. The mass is spread as a thin film on a process film and dried for 15 min at 60-80° C. The dried film is then separated into wafers.

What has been described above are preferred aspects of the present invention. It is of course not possible to describe every conceivable combination of components or methodologies for purposes of describing the present invention, but one of ordinary skill in the art will recognize that many further combinations and permutations of the present invention are possible. Accordingly, the present invention is intended to embrace all such alterations, combinations, modifications, and variations that fall within the spirit and scope of the appended claims.

1. A process for producing a film-shaped preparation for administration of substances to the human or animal body, said preparation being disintegratable in aqueous media and containing at least one water-soluble polymer and at least one gas-forming component which produces a gas upon action of moisture, being in the presence of an aqueous medium or by a temperature change, comprising the steps of:
preparing a coating compound which contains the components of the preparation including said at least one gas-forming component by dissolving or suspending the components in a solvent or suspending agent that is substantially free from water;
spreading said coating compound on a support; and
drying said support.

2. The process according to claim 1, wherein said at least one gas-forming component is selected from the group consisting of carbonates, acids and acid regulators.

3. The process according to claim 2, wherein said at least one gas-forming component is a combination of at least one first component and at least one second component wherein said at least one first component is a carboxylic acid and said at least one second component is selected from the group consisting of sodium hydrogen carbonate, sodium carbonate, potassium carbonate and potassium hydrogen carbonate.

4. The process according to claim 1, further comprising the step of the adding at least one pharmaceutical active substance to said preparation.

5. The process according to claim 1, further comprising the step of adding a flavouring agent to said preparation.

6. A film-shaped preparation disintegratable in aqueous media, for administration of substances to the human or animal body, containing at least one water-soluble polymer, said preparation containing at least one gas-forming component able to produce a gas upon action of moisture, being in the presence of an aqueous medium or by a temperature change.

7. A film-shaped preparation disintegratable in aqueous media, for administration of substances to the human or animal body, containing at least one water-soluble polymer, said preparation containing at least one gas-forming component able to produce a gas upon action of moisture, being in the presence of an aqueous medium or by a temperature change, wherein at least one of said at least one gas-forming component is is in a microencapsulated form.

8. A film-shaped preparation disintegratable in aqueous media, for administration of substances to the human or animal body, containing at least one water-soluble polymer, said preparation containing at least one gas-forming component able to produce a gas upon action of moisture, being in the presence of an aqueous medium or by a temperature change, wherein said preparation has two film layers connected to each other, the first film layer containing a first gas-forming component and further components of the film-shaped preparation, and the second film layer containing a second gas-forming component and further components of the film-shaped preparation, wherein said first and second gas-forming components are reaction partners of a gas-forming reaction.

9. The preparation according to claim 7, wherein said at least one gas-forming component is selected from the group consisting of carbonates, acids and acid regulators.

10. The preparation according to claim 9, wherein said gas-forming components comprise a combination of at least one first component and at least one second component, wherein said at least one first component is a carboxylic acid, and said at least one second component is selected from the group consisting of sodium hydrogen carbonate, sodium carbonate, potassium carbonate and potassium hydrogen carbonate.

11. The preparation according to claim 6, wherein said preparation produces CO₂ or N under action of water, an aqueous medium or moisture.

12. The preparation according to claim 6, wherein said preparation produces an acid environment in the presence of water.

13. The preparation according to claim 6, wherein said preparation disintegrates in the presence of water or an aqueous medium within 1 s to 5 min.

14. The preparation according to claim 6, wherein said preparation is swellable in aqueous media.

15. The preparation according to claim 6, wherein said preparation further contains at least one pharmaceutical active substance.

16. The preparation according to claim 6, wherein said preparation further contains a flavouring agent.

17. The preparation according to claim 6, wherein said preparation comprises at least two layers.

18. The preparation according to claim 6, wherein said preparation has a thickness between 5 μm and 3 mm.

19. The preparation according to claim 6, wherein said preparation is an administration form selected from the group consisting of an oral administrative form, a rectal administrative form and a vaginal administrative form for administration of pharmaceutical active agents.

20. The process according to claim 2, wherein said carbonates are selected from the group consisting of sodium carbonate, ammonium carbonate, magnesium carbonate, potassium carbonate, and hydrogen carbonate, said acids are selected from the group consisting of citric acid, malic acid, acetic acid, lactic acid, fumaric acid, gluconic acid and tartaric acid, and said acid regulators are selected from the group consisting of salts of acetic acid, sodium dihydrogen phosphate, disodium hydrogen phosphate, sodium tartrate and sodium ascorbate.

21. The process according to claim 20, wherein said hydrogen carbonate is sodium hydrogen carbonate.

22. The process according to claim 3, wherein said carboxylic acid is selected from the group consisting of citric acid, malic acid, acetic acid, lactic acid, fumaric acid, gluconic acid and tartaric acid.

23. The process according to claim 5, wherein said flavouring agent is menthol.

24. A process for producing a film-shaped preparation for administration of substances to the human or animal body, said preparation being disintegratable in aqueous media and containing at least one water-soluble polymer and at least one gas-forming component which produces a gas upon action of moisture, being in the presence of an aqueous medium or by a temperature change, comprising the steps of:
preparing a first coating compound which contains a first gas-forming component and further components of the film-forming preparation by dissolving or suspending said components in an aqueous solvent or suspending agent;
preparing a second coating compound which contains said first gas-forming component and further components of the film-shaped preparation by dissolving or suspending said components in an aqueous solvent or suspending agent, said first and said second components being reaction partners of a gas-forming reaction;
spreading the first coating compound on a support;
drying said support to form a first film;
spreading the second coating compound on a support;
drying said support to form a second film; and
laminating said first film and said second film onto each other.

25. The process according to claim 24, wherein said at least one gas-forming component is selected from the group consisting of carbonates, acids and acid regulators.

26. The process according to claim 25, wherein said carbonates are selected from the group consisting of sodium carbonate, ammonium carbonate, magnesium carbonate, potassium carbonate, and hydrogen carbonate, said acids are selected from the group consisting of citric acid, malic acid, acetic acid, lactic acid, fumaric acid, gluconic acid and tartaric acid, and said acid regulators are selected from the group consisting of salts of acetic acid, sodium dihydrogen phosphate, disodium hydrogen phosphate, sodium tartrate and sodium ascorbate.

27. The process according to claim 26, wherein said hydrogen carbonate is sodium hydrogen carbonate.

28. The process according to claim 24, wherein said at least one gas-forming component is a combination of at least one first component and at least one second component, wherein said at least one first component is a carboxylic acid, and said at least one second component is selected from the group consisting of sodium hydrogen carbonate, sodium carbonate, potassium carbonate and potassium hydrogen carbonate.

29. The process according to claim 28, wherein said carboxylic acid is selected from the group consisting of citric acid, malic acid, acetic acid, lactic acid, fumaric acid, gluconic acid and tartaric acid.

30. The process according to claim 24, further comprising the step of the adding at least one pharmaceutical active substance to said preparation.

31. The process according to claim 24, further comprising the step of adding a flavouring agent to said preparation.

32. The process according to claim 31, wherein said flavouring agent is menthol.

33. A process for producing a film-shaped preparation for administration of substances to the human or animal body, said preparation being disintegratable in aqueous media and containing at least one water-soluble polymer and at least one gas-forming component which produces a gas upon action of moisture, being in the presence of an aqueous medium or by a temperature change, or comprising the steps of:
preparing a coating compound which contains the components of the preparation including said at least one gas-forming component by dissolving or suspending the components in a solvent or a suspending agent, wherein at least one of said at least one gas-forming component is present in a microencapsulated form;
spreading said coating compound on a support; and
drying said support.

34. The process according to claim 33, wherein said at least one gas-forming component is selected from the group consisting of carbonates, acids and acid regulators.

35. The process according to claim 34, wherein said carbonates are selected from the group consisting of sodium carbonate, ammonium carbonate, magnesium carbonate, potassium carbonate, and hydrogen carbonate, said acids are selected from the group consisting of citric acid, malic acid, acetic acid, lactic acid, fumaric acid, gluconic acid and tartaric acid, and said acid regulators are selected from the group consisting of salts of acetic acid, sodium dihydrogen phosphate, disodium hydrogen phosphate, sodium tartrate and sodium ascorbate.

36. The process according to claim 35, wherein said hydrogen carbonate is sodium hydrogen carbonate.

37. The process according to claim 33, wherein said at least one gas-forming component is a combination of at least one first component and at least one second component, wherein said at least one first component is a carboxylic acid, and said at least one second component is selected from the group consisting of sodium hydrogen carbonate, sodium carbonate, potassium carbonate and potassium hydrogen carbonate.

38. The process according to claim 37, wherein said carboxylic acid is selected from the group consisting of citric acid, malic acid, acetic acid, lactic acid, fumaric acid, gluconic acid and tartaric acid.

39. The process according to claim 33, further comprising the step of the adding at least one pharmaceutical active substance to said preparation.

40. The process according to claim 33, further comprising the step of adding a flavouring agent to said preparation.

41. The process according to claim 40, wherein said flavouring agent is menthol.

42. The preparation according to claim 13, wherein said preparation disintegrates in the presence of water or an aqueous medium within 1 s to 1 min.

43. The preparation according to claim 42, wherein said preparation disintegrates in the presence of water or an aqueous medium within 1 s to 30 s.

44. The preparation according to claim 16, wherein said flavouring agent is menthol.

45. The preparation according to claim 18, wherein said preparation has a thickness between 10 μm and 1 mm.

46. The preparation according to claim 45, wherein said preparation has a thickness 20 μm and 500 μm.

47. The preparation according to claim 9, wherein said carbonates are selected from the group consisting of sodium carbonate, ammonium carbonate, magnesium carbonate, potassium carbonate, and hydrogen carbonate, said acids are selected from the group consisting of citric acid, malic acid, acetic acid, lactic acid, fumaric acid, gluconic acid and tartaric acid, and said acid regulators are selected from the group consisting of salts of acetic acid, sodium dihydrogen phosphate, disodium hydrogen phosphate, sodium tartrate and sodium ascorbate.

48. The preparation according to claim 47, wherein said hydrogen carbonate is sodium hydrogen carbonate.

49. The preparation according to claim 10, wherein said carboxylic acid is selected from the group consisting of citric acid, malic acid, acetic acid, lactic acid, fumaric acid, gluconic acid and tartaric acid.

50. The preparation according to claim 7, wherein said preparation produces CO₂ or N₂ under action of water, an aqueous medium or moisture.

51. The preparation according to claim 7, wherein said preparation produces an acid environment in the presence of water.

52. The preparation according to claim 7, wherein said preparation disintegrates in the presence of water or an aqueous medium within 1 s to 5 min.

53. The preparation according to claim 52, wherein said preparation disintegrates in the presence of water or an aqueous medium within 1 s to 1 min.

54. The preparation according to claim 53, wherein said preparation disintegrates in the presence of water or an aqueous medium within 1 s to 30 s.

55. The preparation according to claim 7, wherein said preparation is swellable in aqueous media.

56. The preparation according to claim 7, wherein said preparation further contains at least one pharmaceutical active substance.

57. The preparation according to claim 7, wherein said preparation further contains a flavouring agent.

58. The preparation according to claim 57, wherein said flavouring agent is menthol.

59. The preparation according to claim 7, wherein said preparation comprises at least two layers.

60. The preparation according to claim 7, wherein said preparation has a thickness between 5 μm and 3 mm.

61. The preparation according to claim 60, wherein said preparation has a thickness between 10 μm and 1 mm.

62. The preparation according to claim 61, wherein said preparation has a thickness between 20 μm and 500 μm.

63. The preparation according to claim 7, wherein said preparation is an administration form selected from the group consisting of an oral administrative form, a rectal administrative form and a vaginal administrative form for administration of pharmaceutical active agents.

64. The preparation according to claim 8, wherein said at least one gas-forming component is selected from the group consisting of carbonates, acids and acid regulators.

65. The preparation according to claim 64, wherein said carbonates are selected from the group consisting of sodium carbonate, ammonium carbonate, magnesium carbonate, potassium carbonate, and hydrogen carbonate, said acids are selected from the group consisting of citric acid, malic acid, acetic acid, lactic acid, fumaric acid, gluconic acid and tartaric acid, and said acid regulators are selected from the group consisting of salts of acetic acid, sodium dihydrogen phosphate, disodium hydrogen phosphate, sodium tartrate and sodium ascorbate.

66. The preparation according to claim 65, wherein said hydrogen carbonate is sodium hydrogen carbonate.

67. The preparation according to claim 64, wherein said gas-forming components comprise a combination of at least one first component and at least one second component, wherein said at least one first component is a carboxylic acid, and said at least one second component is selected from the group consisting of sodium hydrogen carbonate, sodium carbonate, potassium carbonate and potassium hydrogen carbonate.

68. The preparation according to claim 67, wherein said carboxylic acid is selected from the group consisting of citric acid, malic acid, acetic acid, lactic acid, fumaric acid, gluconic acid and tartaric acid.

69. The preparation according to claim 8, wherein said preparation produces CO₂ or N₂ under action of water, an aqueous medium or moisture.

70. The preparation according to claim 8, wherein said preparation produces an acid environment in the presence of water.

71. The preparation according to claim 8, wherein said preparation disintegrates in the presence of water or an aqueous medium within 1 s to 5 min.

72. The preparation according to claim 71, wherein said preparation disintegrates in the presence of water or an aqueous medium within 1 s to 1 min.

73. The preparation according to claim 72, wherein said preparation disintegrates in the presence of water or an aqueous medium within 1 s to 30 s.

74. The preparation according to claim 8, wherein said preparation is swellable in aqueous media.

75. The preparation according to claim 8, wherein said preparation further contains at least one pharmaceutical active substance.

76. The preparation according to claim 8, wherein said preparation further contains a flavouring agent.

77. The preparation according to claim 76, wherein said flavouring agent is menthol.

78. The preparation according to claim 8, wherein said preparation comprises at least two layers.

79. The preparation according to claim 8, wherein said preparation has a thickness between 5 μm and 3 mm.

80. The preparation according to claim 79, wherein said preparation has a thickness between 10 μm and 1 mm.

81. The preparation according to claim 80, wherein said preparation has a thickness between 20 μm and 500 μm.

82. The preparation according to claim 8, wherein said preparation is an administration form selected from the group consisting of an oral administrative form, a rectal administrative form and a vaginal administrative form for administration of pharmaceutical active agents.
